(19) United States
(12) Patent Application Publication
(10) Pub. No.: US 2008/0038306 A1
(43) Pub. Date: Feb. 14, 2008

(54) Fluidic Tissue Augmentation Compositions and Methods

(76) Inventor: Nathaniel E. David, Los Angeles, CA (US)

Correspondence Address: WILSON SONSINI GOODRICH & ROSATI, 650 PAGE MILL ROAD, PALO ALTO, CA, 94304-1050, US

(21) Appl. No.: 11/685,094

(22) Filed: Mar. 12, 2007

Related U.S. Application Data
(63) Continuation-in-part of application No. 11/276,759, filed on Mar. 13, 2006.

(51) Int. Cl. A61K 31/728; A61F 13/02

(52) U.S. Cl. 424/422; 514/54

(57) ABSTRACT

Compositions and method for augmenting tissue after delivery to localized area. The compositions include a hydrogel and a dermal filler. The hydrogel can polymerize and/or crosslink upon a first trigger event. The dermal filler can also optionally crosslink upon a second trigger event.

### CROSS-REFERENCE

This application is a continuation-in-part of U.S. application Ser. No. 11/276,759, filed on Mar. 13, 2006, entitled “Fluidic Tissue Augmentation Compositions and Methods,” which is incorporated herein by reference in its entirety and to which application we claim priority under 35 USC §120.

### BACKGROUND OF THE INVENTION

Over the past two decades, medical techniques have been developed that allow individuals to significantly improve their physical appearance. Some of these aesthetic medical techniques rely on the use of tissue augmentation materials, such as dermal fillers. Dermal fillers, for example, are agents that are injected into patients to reduce the appearance of facial lines and wrinkles. Unlike botulinum toxin (branded Botox, for example), which is used to paralyze the facial muscles that cause wrinkles, fillers are injected under facial wrinkles and folds to literally fill them in.

Today's fillers suffer from two disadvantages. First, the duration of effect of dermal fillers (i.e., how long an aesthetic correction made via the injection of a dermal filler lasts) is considered too short by both patients and clinicians. For example, patients who have had their nasolabial folds ‘corrected’ by the injection of fillers become anxious when their nasolabial folds begin to reappear after 3-5 months. Because the injection of dermal fillers is painful, sometimes causes bruising, and is inconvenient for patients, it would dramatically improve the commercial appeal of fillers if their duration of effect could be doubled, tripled, or perhaps even quadrupled.

Second, fillers are injected as an amorphous (i.e., shapeless) paste, making it difficult for the physician to engineer certain features into the surface of the human body, such as perfect chin or cheekbone augmentations, as the paste cannot be held in a position/shape adequate to mimic natural chin fat pads or the rounded arcs of human cheek bone structure. While skilled physicians are able to inject fillers to make certain types of facial corrections (e.g., filling of the nasolabial folds, lip augmentations), it is difficult, perhaps even impossible, for clinicians to engineer, say, a perfect chin, as the filler cannot be adequately contoured to render a realistic looking chin shape. As such, a current means to perform a chin augmentation is a surgical procedure (performed under general anesthesia) to slip into the chin area a small plastic implant in the shape of a chin. Similarly, cheekbone augmentation is generally currently achieved not with injectable filler, but rather with the insertion of a plastic implant, requiring use of surgery and general anesthesia. Thus, there is a need for fillers with improved properties such as fillers that hold complex contoured shapes, and those that replace surgical procedures requiring anesthesia.

### SUMMARY OF THE INVENTION

The present invention provides tissue augmentation compositions and methods that are capable of being injected and shaped in situ, and in another aspect, according to a predetermined shape. This overcomes the disadvantages of solid implants, which require surgery, as well as the disadvantages of un-polymerized hydrogel monomer solutions (e.g., not solid enough prior to polymerization to sculpt in situ), and further the disadvantages of the currently available dermal fillers (e.g., lack of persistence, as well as lack of sculptability with any degree of precision). The present materials and methods may also be customized to select for particular in vivo mechanical and persistence properties.

In another aspect, the present invention provides compositions for extending and improving the qualities of the present dermal filler compositions by providing compositions and methods which when combined with the dermal fillers can be used to selectively “tune” or vary the mechanical and persistence properties of the dermal fillers.

In one aspect of the invention, an injectable tissue augmentation composition is provided comprising: a) at least a first fluidic biocompatible moiety capable of selective solidifying upon a first physiologically compatible initiation; b) at least a second fluidic biocompatible moiety optionally capable of selective solidifying upon a second physiologically compatible initiation wherein if the second fluidic biocompatible moiety is capable of said selective solidifying, it is incapable of selective solidifying under said first physiologically compatible initiation; and c) optionally an effective amount of at least one analgesic agent.

In another aspect of the invention, an injectable tissue augmentation composition is provided comprising: a hydrogel forming moiety which is capable of selective solidifying under physiologic conditions in the presence of a wavelength of light capable of penetrating through human skin of a thickness of between about 1-2 mm, optionally in the presence of a plastic mold; and, which is selectively degradable in situ; and a second moiety selected from the group consisting of a collagen containing moiety, a collagen-derivative containing moiety; a hyaluronic acid containing moiety, a hyaluronic acid derivative containing moiety, a chondroitin containing moiety, and a chondroitin derivative containing moiety.

In another aspect of the invention, the use of a composition is provided comprising a ) at least a first fluidic biocompatible moiety capable of selective solidifying upon a first physiologically compatible initiation; b) at least a second fluidic biocompatible moiety optionally capable of selective solidifying upon a second physiologically compatible initiation wherein if the second fluidic biocompatible moiety is capable of said selective solidifying, it is incapable of selective solidifying under said first physiologically compatible initiation; and c) optionally an effective amount of at least one analgesic agent, in the manufacture of a medicament for therapeutic and cosmetic augmentation of tissue.

In a further aspect of the invention, the use of a moldable tissue augmentation composition is provided comprising at least one hydrogel, at least one dermal filler, and optionally at least one analgesic agent in the manufacture of a medicament for therapeutic and cosmetic augmentation of tissue in a predetermined shape.

In another aspect of the invention, the use of a tissue augmentation material is provided comprising at least one hydrogel, at least one dermal filler, and optionally at least one analgesic agent in the manufacture of a medicament for the therapeutic and cosmetic alteration of the shape of a nose bridge to a predetermined shape. In another aspect of the invention, the use of a tissue augmentation material is provided, comprising at least one hydrogel, at least one dermal filler, and optionally at least one analgesic agent, in the manufacture of a medicament for therapeutic and cosmetic facial sculpting

In a third aspect of the invention a photofiller is provided consisting essentially of a hydrogel and a hyaluronic acid containing dermal filler.

In another aspect of the invention a kit is provided comprising a first prefilled syringe containing a photopolymerizing hydrogel forming moiety; and a second prefilled syringe containing a dermal filler, and optionally a transparent mold wherein the inner surface of the mold is in the shape of a body part.

In yet another aspect of the invention a method for augmenting tissue in a predetermined shape is provided comprising injecting a moldable tissue augmentation composition to the tissue for which augmentation is desired; applying a mold externally to skin covering the tissue for which augmentation is desired, wherein an inner concave surface of the mold is in a predetermined shape so that the tissue augmentation material will bring the skin into contact with the inner concave surface of the mold; and, increasing the solidity of the tissue augmentation material whereby the tissue augmentation material holds the shape of the inner concave surface of the externally applied mold.

In another aspect of the invention a method for altering the shape of a nose bridge to a predetermined shape comprising injecting a moldable tissue augmentation composition to the nose bridge in the presence of an inner surface of a mold of the predetermined shape; and increasing the solidity of the tissue augmentation composition whereby the tissue augmentation composition maintains the shape of the inner surface of the mold.

In a further aspect of the invention a method for facial sculpting comprising predetermining the final shape of the sculpted face by using digital three dimensional information to prepare a mold having an inner surface comprising the precise dimensions of the final sculpted face; injecting tissue augmentation material capable of increasing in solidity in situ using physiologically compatible initiation using the mold as a guide to form the shape of the sculpted face; applying physiologically compatible initiation to increase the solidity of the tissue augmentation material to maintain the shape of the inner surface of the mold.

In yet another aspect of the invention, a method of augmenting tissue is provided, having the steps of injecting subdermally a composition of the invention which is capable of solidifying upon exposure to physiologically compatible initiation; applying external pressure over the injected composition to determine the shape of the augmented tissue; and applying the physiologically compatible initiation to solidify the composition.

In another aspect of the invention a method of augmenting tissue comprising injecting subdermally a composition comprising an uncrosslinked hydrogel containing moiety which is adapted to crosslink upon exposure to light and a hyaluronic acid moiety; and exposing the composition to light to crosslink the hydrogel.

In yet another aspect of the invention, a method of producing a cosmetic or therapeutic medicament is provided wherein an uncrosslinked hydrogel containing moiety, which is adapted to crosslink upon exposure to light and a hyaluronic acid moiety are packaged in separate containers in single use dosages.

In another aspect of the invention a method of preparing and using a mold to predetermine the shape of the tissue augmentation is provided. In some embodiments of the invention, the mold is pre-prepared using a computer program capable of transmitting three dimensional coordinates of the predetermined shape to a device which prepares a tangible mold reflecting the three dimensional coordinates. In some embodiments the mold is applied to the skin before applying the moldable tissue augmentation material. In some embodiments the mold is applied to the skin at the same time as the moldable tissue augmentation material is applied.

In another aspect of the invention, a business method contemplates providing a treatment service in exchange for a service fee. The service can be provided directly to the patient by a health care provider. The business method contemplates a computer-based method of providing a customized tissue augmentation kit for a provider of tissue augmentation services.

The present invention also includes a business method for providing a customized tissue augmentation kit for a particular patient including transmitting to a receiving computer a computer file containing three dimensional coordinates of the desired shape of tissue-augmented area of a particular patient; and a computer file containing the desired mechanical and persistence properties of the tissue augmentation material; wherein such information is used to prepare a customized tissue augmentation kit for use by a provider on the particular patient. The kit so provided contains a mold for the predetermined tissue augmented shape, an injectable tissue augmentation material capable of selectively solidifying in situ and having preselected persistence and mechanical properties in accordance with the computer file so transferred.

In some embodiments of the invention, the first fluidic biocompatible moiety is a hydrogel forming moiety. In other embodiments of the invention, the hydrogel forming moiety comprises a natural or a synthetic moiety and may be monomers or polymers. In other embodiments, the hydrogel forming moiety, or photopolymerizing hydrogel forming moiety is selected from the group consisting of poly(lactic acid) (PLA), poly(glycolic acid) (PGA), and their copolymers, poly(lactic-co-glycolic acid) (PLGA), Polyethylene glycol diacrylate (PEG-DA), poly(ethylene oxide) (PEO), Pluronic, (PEO-PPO-PEO), poly(ethylene oxide)diacrylate (PEODA), polyalkylene oxides, polyethylene glycols, polyethylene oxides, partially or fully hydrolyzed polyvinylalcohols, poly(vinylpyrrolidone), poly(t-ethyloxazoline), poly(ethylene oxide)-co-poly(propylene oxide) block copolymers (poloxamers and meroxapols), propylene glycol, trimethylene glycols, mono-, di- and tri-polyoxyethylated glycerol, mono- and di-polyoxy-ethylated propylene glycol, and mono- and di-polyoxyethylated trimethylene glycol, polyoxyethylated sorbitol, polyoxyethylated glucose, polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), polymaleic acid, polyacrylamide, poly(methacrylamide), poly(dimethylacrylamide), poly(N-isopropyl-acrylamide), poly(olefinic alcohol), poly(N-vinyl lactams), poly(N-vinyl caprolactam), polyethylene glycol/poly(N-isopropylacrylamide); polyoxazolines, poly(methyloxazoline), poly(ethyloxazoline), polyvinylamines, polyacrylamide (PAA), poloxamines, carboxymethyl cellulose, and hydroxyalkylated celluloses.

In other embodiments, the hydrogel forming moiety comprises a derivatized polyethylene glycol monomer. In some of the embodiments of the invention, hydrogel forming moiety comprises polyethylene glycol derivatized with diacrylate. In other embodiments of the invention, the hydrogel forming moiety or photopolymerizing hydrogel forming moiety is chosen from the group consisting of glycosaminoglycans, polypeptides, proteins, polysaccharides, and carbohydrates. In yet other embodiments of the invention, the hydrogel forming moiety is selected from the group consisting of hyaluronic acid, chondroitin sulfate A, chondroitin sulfate C, dermatan sulfate, keratan sulfate, keratosulfate, chitin, chitosan, polysucrose, dextran, heparin sulfate, heparin, alginate, gelatin, collagen, albumin or ovalbumin, elastins, laminins, gelatins, and fibronectins. In some embodiments of the invention the first biocompatible moiety comprises more than one fluidic biocompatible moiety capable of selective solidifying upon a first physiologically compatible initiation.

In some embodiments of the invention, the second moiety is a dermal filler. In some embodiments of the invention, the second fluidic biocompatible moiety or dermal filler comprises an extracellular matrix protein, an extracellular matrix polysaccharide or an extracellular proteoglycan. In other embodiments of the invention the at least a second fluidic biocompatible moiety or dermal filler is selected from the group consisting of collagens, hyaluronic acid, elastins, laminins, fibronectins, chondroitin sulfate, keratin sulfate, hyaluronic acid, heparan sulfate, chondroitin sulfate, amylose, maltodextrin, amylopectin, starch, dextran, heparin, dermatan sulfate, dextran sulfate, pentosan polysulfate, and chitosan. In some of the embodiments of the invention, the at least second fluidic biocompatible moiety or dermal filler is hyaluronic acid. In other embodiments of the invention, the second fluidic biocompatible moiety or dermal filler is selected from the group consisting of a polyamino acid containing moiety, a polysaccharide moiety, and a glycoprotein moiety. In some embodiments, more than one of these materials are included in the compositions of the invention.

In some of the embodiments of the invention, the first fluidic biocompatible moiety selectively solidifies in the presence of light. In some embodiments of the invention, the hydrogel selectively solidifies in the presence of light. In some embodiments the second fluidic biocompatible moiety solidifies in the presence of light. In other embodiments of the invention, the dermal filler solidifies in the presence of light. In yet other embodiments of the invention, increasing the solidity of tissue augmentation material is initiated by a light source. In some embodiments of the invention, the light is selected from the group consisting of ultraviolet and visible light. In other embodiments of the invention, the light has a wavelength of about 400-550 nm. In some embodiments of the invention, the exposure of light is transdermal. In some other embodiments of the invention, light is applied subdermally.

In some embodiments of the invention, the moldable tissue augmentation material comprises a hydrogel and a hyaluronic acid. In some embodiments of the invention, the moldable tissue augmentation material comprises a hydrogel and a dermal filler. In other embodiments of the invention, the tissue augmentation composition further comprises an initiator of crosslinking. In other embodiments of the invention, the chemical initiator initiates thermal, photo, redox, atom transfer, cationic, anionic, coordination, ring opening and/or metathesis polymerization. In some embodiments of the invention, the chemical initiator is selected from the group consisting of 4-benzoylbenzoic acid, [(9-oxo-2-thioxanthanyl)-oxy]acetic acid, 2-hydroxy thioxanthone, vinyloxymethylbenzoin methyl ether; 4-benzoylbenzoic acid, [(9-oxo-2-thioxanthanyl)-oxy]acetic acid, 2-hydroxy thioxanthone, vinyloxymethylbenzoin methyl ether; acridine orange, ethyl eosin, eosin Y, Eosin B, erythrosine, fluorescein, methylene green, methylene blue, phloxime, riboflavin, rose bengal, thionine, xanthine dyes, 4,4′azobis(4-cyanopentanoic)acid and 2,2-azobis[2-(2-imidazolin-2-yl)propane]dihydrochloride, 4,4′azobis(4-cyanopentanoic) acid and 2,2-azobis[2-(2-imidazolin-2-yl)propane]dihydrochloride.

In some embodiments of the invention the injectable tissue augmentation composition comprises a hydrogel and a dermal filler. In some embodiments of the invention, the first fluidic biocompatible moiety selectively solidifies in the presence of a chemical initiator. In some embodiments of the invention, the first fluidic biocompatible moiety selectively solidifies in the presence of a thermal initiator. In some embodiments of the invention, the hydrogel selectively solidifies in the presence of a chemical initiator. In some embodiments the second fluidic biocompatible moiety solidifies in the presence of a chemical initiator. In other embodiments of the invention, the dermal filler solidifies in the presence of a chemical initiator.

In yet other embodiments of the invention the hydrogel forming moiety is derivatized to selectively solidify in the presence of a light wavelength which substantially penetrates mammalian skin below the epidermis; and, the second fluidic biocompatible moiety is substantially incapable of selective solidifying in the presence of a light wavelength which penetrates human skin. In some embodiments of the invention, a physiologically compatible initiation is light.

In some embodiments of the invention, the hydrogel forming moiety is selectively degradable in situ. In some embodiments of the invention, the hydrogel forming moiety is capable of photoinitiated solidifying under physiological conditions and is capable of selective degradation in situ. In some embodiments of the invention, the hydrogel forming moiety or photopolymerizing hydrogel forming moiety is selectively degraded in situ by administering a biologically compatible degradation agent. In some embodiments of the invention, the selective degradation is performed to reverse a tissue augmentation. In other embodiments, the hydrogel forming moiety is selectively degraded in situ over time. In yet other embodiments of the invention, the second fluidic biocompatible moiety is selectively degraded in situ by administering a biologically compatible degradation agent. In other embodiments of the invention, the tissue augmentation material is a hydrogel forming composition capable of controllable degradation.

In some of the embodiments of the invention, the injectable tissue augmentation composition or moldable tissue augmentation material further comprises one or more analgesic agents. The analgesic agents are selected from the group consisting of lidocaine, mepivacaine, bupivacaine, procaine, chloroprocaine, etidocaine, prilocaine dyclonine, hexylcaine, procaine, cocaine, ketamine, morphine, pramoxine, propophol, phenol, naloxone, meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin(diacetylmorphine), hydrocodone(dihydrocodeinone), hydromorphone(dihydromorphinone), levorphanol, meptazinol, methadone, metopon(methyldihydromorphinone), nalbuphine, oxycodone(dihydrohydroxycodeinone), oxymorphone(dihydrohydroxymorphinone), phenadoxone, phenazocine, remifentanil, tramadol, tetracaine, and their pharmaceutically acceptable salts. In other embodiments of the invention, the analgesic agent is lidocaine or its pharmaceutically acceptable salts. In yet other embodiments of the invention, the tissue augmentation composition comprises more than one analgesic agent

In some embodiments of the invention, the compositions comprise one or more of the group consisting of particulate matter, therapeutic moieties, medical devices, electronic devices, living cells, pigments, enzyme inhibitors, and enzymes.

In some of the embodiments of the invention, the ratio of the first fluidic biocompatible moiety and the second fluidic biocompatible moiety in the composition is in the range of about 1:2 to about 1:10 w/w. In some of the embodiments of the invention, the ratio of the hydrogel and the dermal filler is in the range of about 1:2 to about 1:10 w/w. In some of the embodiments of the invention, the ratio of components in the composition are altered to change the solidity of the augmented tissue. In some other embodiments of the invention, the tissue augmentation compositions can comprise more than 1, 2, 3, 4, 5, 6, 7, 8, 9, or 10 different monomers. In the methods of the invention, the time to achieve increased solidification is less than 10, 9, 8, 7, 6, 5, 4, 3, 2 minutes and may be less than one minute.

In some other embodiments of the invention, a photofiller consisting essentially of a hydrogel and a hyaluronic acid containing dermal filler is provided. In yet other embodiments of the invention, the tissue augmentation composition is used in the manufacture of a medicament for cosmetic and therapeutic tissue augmentations.

The tissue to be augmented with the compositions and methods of the invention include dermal tissue augmentation, filling of lines, folds, wrinkles, minor facial depressions, cleft lips and the like, especially in the face and neck; correction of minor deformities due to aging or disease, including in the hands and feet, fingers and toes; augmentation of the vocal cords or glottis to rehabilitate speech; dermal filling of sleep lines and expression lines; replacement of dermal and subcutaneous tissue lost due to aging; lip augmentation; filling of crow's feet and the orbital groove around the eye; breast or penile augmentation; chin augmentation; augmentation of the cheek and/or nose; filling of indentations in the soft tissue, dermal or subcutaneous, due to, e.g., overzealous liposuction or other trauma; filling of acne or traumatic scars and rhytids; filling of breasts and/or buttocks; filling of nasolabial lines, nasoglabellar lines and infraoral lines, augmenting bone, and augmenting nasal cartilage.

Various aspects of the present invention are provided including methods, compositions including kits, as well as computer based methods and systems. Although this Summary of the Invention has set forth major aspects of the present invention, set forth below are various additional aspects and embodiments provided.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

I Terminology

As used herein, the terms “tissue augmentation” refers generally to the addition of matter to cellular or a cellular body structures, such as adipose tissue, connective tissue, muscle tissue, cartilage tissue or any combination of tissues. Such tissue may be ordinarily considered soft tissue (e.g., muscle or fat) or hard tissue (e.g., bone or cartilage). The term “dermal filler” as used herein is a type of tissue augmentation material which is generally used in the dermis area, such as below the epidermis or above the hypodermis, and as such may be injected subcutaneously, hypodermically or intradermally, or some combination.

The term “photofiller” as used herein refers to tissue augmentation compositions that can be solidified in situ using light. As described more fully herein, this preferably involves hydrogels derivatized for light-initiated polymerization. Depending on the wavelength of light used and the depth to which the tissue augmentation is injected beneath the outer surface of the skin, the present “photofiller” compositions may be polymerized transdermally, by shining light (e.g., UV wavelengths or IR wavelengths) through the skin (i.e. light source can be external or internal to skin), thereby initiating the polymerization.

The term “cross-link” may be a covalent or non-covalent bond that connects units in a complex chemical molecule (as a protein). Cross linkages may be inter or intra-molecular. In situ cross-linking herein denotes cross-linking occurring at the site of application, for example, the injection site of the fluidic tissue augmentation composition.

The term “hydrogel” refers to a class of polymeric materials that swell in water and do not dissociate or depolymerize in water. They may be synthesized from water-soluble monomers or monomers mixed with polymers and are substantially water insoluble. Hydrogels may be cross-linked to form an interpenetrating network. A “hydrogel precursor” denotes a hydrogel material that is not cross-linked so that the monomeric or polymeric material is not in a form that is substantially water insoluble.

Unless otherwise indicated, all numbers expressing quantities of ingredients, properties such as molecular weight, reaction conditions, and so forth used in the specification and claims are to be understood as being modified in all instances by the term “about.”

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed.

Groupings of alternative elements or embodiments of the invention disclosed herein are not to be construed as limitations. Each group member may be referred to and claimed individually or in any combination with other members of the group or other elements found herein. It is contemplated that one or more members of a group may be included in, or deleted form, a group. When any such inclusion or deletion occurs, the specification is herein deemed to contain the group as modified unless specifically noted.

II Augmentation And Facial Sculpting

The present invention provides compositions and methods for use in tissue augmentation for both therapeutic and cosmetic applications. Injectable fluidic biocompatible materials are disclosed that are suitably formulated for injection into patients as a localized soft paste, which are then crosslinked in-situ to provide polymerized tissue augmentations which are resistant to metabolic destruction. The injected compositions are crosslinked using either photo or chemical activation. The resultant crosslinked compositions form materials with either similar mechanical properties to the injected soft paste, or alternatively, more hardened mechanical properties. These crosslinked materials permit clinicians to exercise a greater level of control to engineer specifically shaped and contoured features. In some of the methods provided, the materials are molded in-situ into a desired shape after injection. In some of the methods provided, a mold is prepared of the body part to guide the contouring. These resistant tissue augmentations result in aesthetic corrections which possess superior longevity in-vivo. Business methods to utilize the methods, molds, and compositions provided are included.

A. Tissue Augmentation Compositions

The compositions herein can include one or more fluidic biocompatible moieties such as silicone and functionalized silicon polymers, hydrogels, hyaluronic acid, collagen, polylactic acid, hydroxylapatite suspensions, collagens, as well as cells and tissue that are autographed or xenographed or transplanted, such as cadaveric and autologous fat cells or any dermal fillers described in Table 1 below. The materials can be used for augmenting the lips or nasolabial folds. A permanent and immobile substance may be appropriate for correcting an iatrogenic scar; correcting rhytids (skin wrinkles) that change with age,

Hydrogels are typically composed of three-dimensional networks formed by the cross-linking of water-soluble monomers into water-insoluble polymer networks. Hydrogels are of particular interest in the field of tissue engineering because of their tissue-like water content, which allows for nutrient and waste transport.

The present invention contemplates compositions comprising, consisting essentially of, or consisting of dermal fillers such as any known fillers (e.g., Restylane™, which is composed of hyaluronic acid) with solutions of monomers (e.g., PEG-diacrylate or hydroxy ethylene methacrylate) that can be polymerized into hydrogels. In some cases, polymerization is initiated upon exposure to light. These hybrid materials have the identical mechanical/persistence properties of the original filler (e.g., Restylane™) before injection, but have enhanced mechanical (e.g., harder) and persistence (i.e., longer) properties after polymerization.

Initiation of the cross-linking reaction can occur using various materials and methods, such as chemically reactive or temperature-sensitive agents. Photoinitiated cross-linking provides a fast and efficient method to cross-link the injected fluidic material to form a hydrogel inside the body, with significant temporal and spatial control, thus creating a material with more rigid mechanical properties only after exposure to light of a specific wavelength. For example, chondroitin sulfate, which is composed of repeating disaccharide units of glucuronic acid and N-acetylgalactosamine with a sulfate (SO4) group and a carboxyl (COOH) group on each disaccharide, can be modified with (meth)acrylate groups and further with an agent to allow cross-linking and thus polymerization in the presence of a photoinitiator.

In some cases photoinitiated cross-linking occurs in situ. This allows for hydrogel formation to be created with minimally invasive systems for biomaterial implantation. Photoinitiated polymerization for tissue augmentation is advantageous in that the liquid-like composition can be polymerized and solidified via cross-linking the injected monomers after it is injected into the dermis. Transdermal photoinitiation, i.e. shining light through the skin, is one way to cross link photo-activatable monomers into polymers in situ.

Thus the tissue augmentation compositions of the present invention can have one or more of these properties:

(a) a sufficiently liquid consistency, preferably moldable or malleable, before polymerization to enable placement within tissues without surgery, e.g., via injection,

(b) the property of being capable of selectively increasing solidity (e.g., polymerizing or crosslinking) in situ under physiologic conditions so that the selected shape is maintained for a controllable period of time; and,

(c) the property of having tunable (i.e., controllable) in vivo persistence and mechanical properties.

Other qualities that are desirable in a tissue augmentation composition is that every component in the composition has an acceptable level of biocompatibility. Although there may be trace or some degree of a non-biocompatible response by use of the present compositions, the degree of acceptable non-biocompatible response can be determined by the medical provider, e.g., treating physician. For example, a particularly efficacious photoinitiator cross-linking agent may be selected so that hydrogel (see below) polymerization is accomplished without undue exposure to ultraviolet light, even though that particular cross linking agent may produce non-biocompatible effects, if the benefits of using that reagent outweigh the possible harm of undue exposure to ultraviolet light.

More specifically, the tissue augmentation compositions of the present invention are injectable and comprise a) at least a first fluidic biocompatible moiety capable of selective solidifying upon a first physiologically compatible initiation; and b) at least a second fluidic biocompatible moiety optionally capable of selective solidifying upon a second physiologically compatible initiation wherein if the second fluidic biocompatible moiety is capable of said selective solidifying, it is incapable of selective solidifying under said first physiologically compatible initiation. The compositions may further comprise an effective amount of at least one analgesic agent. In some embodiments of the present invention, the composition selectively solidifies in the presence of light.

The present compositions are prepared using at least two major components: a first component comprising a polymeric backbone (or covalently linked polymeric backbones) (e.g., a hydrogel network), and a second component (e.g., a dermal filler) that can be composed of a different material than the polymeric backbone. The second component is entrapped within, but not chemically cross-linked to, the first component (e.g., hydrogel network). The second component may optionally be self-cross-linked, but is not covalently cross-linked to the first component (e.g., hydrogel network).

One may selectively increase the persistence of present dermal filler materials without altering the dermal filler chemical integrity. One can also make the hydrogel backbone (for example) controllably solidified or fluidic or biodegradable, so that under the certain conditions (e.g., temperature, light, enzymes, see below), the augmentation is reversed. In this way, if the medical provider desires to “redo” the procedure or the patient's bone structure changes and the previous augmentation is no longer appropriate, the material can be safely absorbed without the need for surgical removal.

The tissue augmentation compositions can comprise 1 or more than 1, 2, 3, 4, 5, 6, 7, 8, 9, or 10 different monomers. Because it may be preferable not to have the backbone (e.g., hydrogel) non specifically crosslink with, for example, hyaluronic acid or other more liquid dermal filler component, crosslinking moieties selected wherein each is activated to crosslink under different conditions.

The ratio of polymeric backbone (e.g., hydrogel) to dermal filler in the compositions of the present invention may be less than 1:1 in that there is a small amount of hydrogel containing a larger amount of dermal filler, on a w/w, v/v or mole/molar basis. In some embodiments, the ratio of polymeric backbone (e.g., hydrogel) to dermal filler is less than 1:2, 1:3, 1:4, 1:5, 1:6, 1:7, 1:8, 1:9, or 1:10 on a w/w, v/v or mole/molar basis. In other cases it is more than 1:2, 1:3, 1:4, 1:5, 1:6, 1:8, 1:9 or 1:10 on a w/w, v/v, or mole/molar basis.

1. Hydrogel Components

Hydrogels are water-insoluble three-dimensional networks that are formed by the cross-linking of water-soluble monomers. The cross-linking of the water soluble monomers into a water insoluble polymer allows the hydrogel to “swell” and topologically trap other compositions, such as the dermal filler composition, thereby forming an interpenetrating covalent network in and around the dermal filler. For the present purposes, the hydrogels of the present invention may be formed in situ with water from the surrounding tissue.

A variety of monomers and polymers, and combinations thereof, can be used to form biocompatible hydrogels. Either synthetic or natural monomers/polymers may be used.

Useful synthetic materials are poly(lactic acid) (PLA), poly(glycolic acid) (PGA), and their copolymers, poly(lactic-co-glycolic acid) (PLGA). Additionally, monomers such as PEG-DA can be mixed with any of the common dermal filler agents listed above (e.g., 0.1%-10% of the filler by mass could be mixed with PEG-DA monomer). Polyethylene glycol diacrylate monomers (“PEG-diacrylate”) may be used as a starting point for selectively customizing the mechanical and persistence (durability) properties of the tissue augmentation material. Synthetic hydrogels include poly(ethylene oxide)(PEO) based polymers and can be found as copolymers such as Pluronic, a triblock copolymer of poly(ethylene oxide) and poly(propylene oxide) (PEO-PPO-PEO), or derivatized to be capable of photoinitiated cross-linking, such as poly(ethylene oxide)diacrylate (PEODA). Some examples of useful synthetic polymers include, but are not limited to: polyalkylene oxides, polyethylene glycols, polyethylene oxides, partially or fully hydrolyzed polyvinylalcohols, poly(vinylpyrrolidone), poly(t-ethyloxazoline), poly(ethylene oxide)-co-poly(propylene oxide) block copolymers (poloxamers and meroxapols), polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers such as polyethylene glycol/poly(N-isopropylacrylamide)thereof; polyoxazolines, including poly(methyloxazoline) and poly(ethyloxazoline); polyvinylamines, polyacrylamide (PAA), poloxamines, carboxymethyl cellulose, and hydroxyalkylated celluloses such as hydroxyl-ethyl cellulose and methylhydroxypropyl celluloses. One may use various combinations, and further various chemically modified forms or derivatives thereof. Functionalized chondroitin sulfate may be used.

Natural monomers may include glycosaminoglycans such as hyaluronic acid, chondroitin sulfate A, chondroitin sulfate C, dermatan sulfate, keratan sulfate, keratosulfate, chitin, chitosan, and derivatives thereof. Therefore, while not exhaustive, examples of natural monomers or polymers which may be used in hydrogel preparation include: polypeptides, polysaccharides or carbohydrates such as polysucrose, hyaluronic acid, dextran, heparin sulfate, chondroitin sulfate, heparin, or alginate, and proteins such as gelatin, collagen, albumin or ovalbumin or copolymers or blends thereof. Celluloses include cellulose and derivatives, dextrans include dextran and similar derivatives. Extracellular matrix proteins, such as collagens, elastins, laminins, gelatins, and fibronectins include all the various types found naturally (e.g., Collagen I-IV) as well as those same collagens as produced by and purified from a recombinant source. Fibrin, a naturally occurring peptide important for its a role in wound repair in the body, and alginate, a polysaccharide derived from seaweed containing repeating units of mannuronic and guluronic acid, may also be used. One may use various combinations, and may include chemically modified forms, mimetics, or derivatives thereof. For proteins, one may use recombinant forms, analogs, forms containing amino acid mimetics, and other various protein or polypeptide-related compositions. The hydrogels of the present invention may be derivatized appropriately to provide crosslinking capability under preselected conditions. Specific derivatization is discussed further below.

2. Dermal Filler Components

One or more dermal filler moiety(ies) maybe selected from among those currently used in humans, e.g., collagens, hyaluronic acid-containing compositions, such compositions containing particulate matter (hydroxylapatite or other calcium containing particles). One useful dermal filler is a hyaluronic acid containing moiety, such as Restylane™-branded material.

The dermal filler moieties may be selected from or an extracellular matrix polysaccharide (or proteoglycan).among those containing an extracellular matrix component, such as an extracellular matrix protein The dermal filler moiety may be a combination of dermal fillers, such as a combination containing a recombinant collagen and a recombinant hyaluronic acid.

More broadly, extracellular matrix proteins may be naturally found or made by recombinant means, and thus may have various moieties incident to the methods, recombinant DNAs, and organisms so producing (e.g., an N-terminal methionyl residue or glycosylation pattern incident to the producing organism, such as yeast). Extracellular matrix proteins include the structural proteins collagen and elastin. A number of different collagen types have been found in humans (19 different types); collagen types I-IV are most well characterized. For use in humans, typically a biologically compatible collagen will be used, preferably a human collagen, and more preferably a recombinant human collagen. The recombinant human collagen may have all or part of the amino acid sequence of a naturally occurring human collagen. Cell binding or adhesive extracellular matrix proteins include laminin and fibronectin. Preferred for biocompatibility in humans, and limited disease transmission, are recombinant proteins containing all or part of the amino acid sequence of a naturally occurring human protein. The extracellular matrix protein may be engineered to optimize a desired characteristic, such as predetermined degradation, gellation, consistency or other persistence or mechanical properties. One may engineer in an extracellular matrix binding functionality, such as an “RGD” moiety or mimetic or related functional moiety.

The dermal filler moiety may also be composed of elements derived from the extracellular matrix polysaccharides, including hyaluronic acid, heparan sulfate, chondroitin sulfate, and keratin sulfate, amylose, maltodextrin, amylopectin, starch, dextran, heparin, dermatan sulfate, keratan sulfate, dextran sulfate, pentosan polysulfate, and chitosan and their respective derivatives. The polysaccharide may be in the form of a proteoglycan, or sugar moiety bound to a protein moiety. One or more polysaccharides or proteoglycans may be used together, and further, one or more may be used in combination with one or more extracellular matrix proteins.

3. Additional Components Of Tissue Augmentation Compositions

a. Particulate matter. Particulate matter may be admixed for lengthening the duration of aesthetic corrections resulting from tissue augmentation, as well as being useful as a bulking or filling density agent. Materials giving structural strength, and durability, such as calcium containing materials (e.g., hydroxyl apatite) or carbohydrate containing materials, (e.g., chitin or chitosan) may be included as particulate matter. Such particulate matter may add to the persistence of the polymeric material within the dermis. One may mix solid or semi-solid microparticles, such as silicone or lipid microparticles, to obtain a desired consistency.

Rigid gelatinous material may also essentially form particulate materials. For example, aerogel is a solid-state substance similar to gel where the liquid component is replaced with gas. The result is an extremely low density solid with several remarkable properties, most notably its effectiveness as an insulator. Aerogel is typically composed of 99.8% air (or vacant space) with a typical density of 3 mg/cm³. Aerogel is extremely delicate, in that pressure causes the material to shatter like glass. Depending on the manufacturing technique, however, an aerogel composition may hold over 2000 times its own weight. Typically, an aerogel will have a dendritic microstructure formed by spherical particles fused together into clusters. These clusters together form three-dimensional highly porous structures of fractal-like chains with pores typically smaller than 100 nanometers. The average size and density of pores can be controlled during the manufacturing process. Aerogels by themselves are hydrophilic, but chemical treatment of their surface can make them hydrophobic. Thus, a biocompatible aerogel may be used in conjunction with the present tissue augmentation materials as a structural material. In the alternative, or additionally, an aerogel composition which is extremely pressure sensitive (the common term of art is ‘friable’) may be suitable for selective degradation of the tissue augmentation material in that high pressure may cause shattering, which can be released into the surrounding tissue with degradation of the overall interpenetrating network, for example.

b. Therapeutic moieties. The material may be polymerized in situ while containing bioactive moieties, such as therapeutic moieties. Among those contemplated are those moieties which are similar to naturally occurring human or animal moieties, or those which are totally synthetic and do not occur in nature. For example, naturally occurring or synthetic extracellular matrix-effecting moieties may be included, such as metalloproteinases, metalloproteinase inhibitors, extracellular matrix proteins, and adhesion-related molecules, in order to promote various therapeutic interventions, e.g., for example, osteogenesis. Naturally occurring or synthetic growth factors, such as human or animal growth hormones, fibroblast growth factor, epidermal growth factors, kerotinocyte growth factors, bone cell growth affecting materials, or other moieties may be included depending on the therapeutic need therefore. Such therapeutic moieties may contain all or part of the natural human (or other animal) amino acid sequence, and may include synthetic moieties, such as peptidomimetic regions.

c. Medical or Other Devices. Apart from chemical moieties, devices may be incorporated into the polymeric material prior to polymerization. In one aspect, the present invention contemplates incorporation of miniature devices, such as pumps, such as radio frequency devices for tracking or identification, nanosensors to determine body component levels, such as insulin levels or blood sugar levels, or other “motes” which act as sensors to communicate information about the local environment to a receiver.

For example, a micro device with reservoirs for one or more drugs can be localized within fluidic tissue augmentation material before or after placement within the tissue to be augmented, but prior to cross-linking as described herein. Release of the drug can be triggered by communication with a programmed wireless receiver. Preprogrammed microprocessors, remote controls, or biosensors can be used to open micro reservoirs to achieve intricate chemical release models.

Tracking devices include insertion of radio-labeled “tags” for animal management, such as radio-frequency controlled labels (e.g., “RFID” or radio frequency ID”) which can be read or tracked at a remote location. Thus, the present invention includes compositions and methods including a “tag” such as a RFID. Methods include injecting an animal with the fluidic tissue augmentation composition, such as a hydrogel capable of cross-linking in situ, containing such RFID or other identification tag, and solidifying the tissue augmentation in situ. As above, transdermal photoinitiation may be most useful. Use of an RFID may be for convenience (e.g., for an individual identity marker for security purposes), or for maintaining security for vulnerable individuals, such as the infirm, the elderly or children. Veterinary applications may be of value, such as pet ID or tracking, or for agricultural purposes, such as identifying herd animals.

Encapsulating devices such as the RFID device within the present compositions using the present methods may aid in avoiding deleterious physiological response, such as immune or allergic response to device materials, such as metals or alloys.

d. Cell Free or With Cells. The present compositions and methods may be free of living cells, or may contain living cells prior to polymerization. For example, a patient's autologous cell may be premixed with the fluidic tissue augmentation material. Upon injection and cross-linking, the cell will be immobilized within the cross-linked polymer network. This type of “scaffolding” may or may not be for permanent placement of the tissue augmentation materials, but may be temporary support until the cells are integrated into the endogenous tissue.

The present compositions and methods may optionally be used for tissue generation in situ. Tissue to be generated includes fat, muscle or cartilage. The three-dimensional structural support that an in situ polymerized biomaterial provides may provide an environment suitable for tissue regeneration. Cartilage tissue may also be generated using the present compositions and methods. Cartilage tissue has been the subject of tissue engineering in situ.

For example, one may desire tissue augmentation to reshape the nose using a mold. Using tissue augmentation material as scaffolding for integration of cartilage precursor cells so that a portion of the nasal cartilage is cellularly integrated with the newly formed tissue augmentation material may provide a non-invasive method of cosmetic or reconstructive rhinoplasty.

Under some circumstances, the present composition may result in cell binding in vivo, so that, although cells of an ex-vivo source are not co-administered as part of the injectable composition, in vivo, cells adhere and grow on the injectable composition.

e. Other Components. Other components may be part of the tissue augmentation compositions and methods. These other components may be added concomitantly, or admixed into the liquid polymer upon injection, or administered after injection of the liquid polymer but prior to in situ cross-linking, or administered after the cross-linking.

Analgesics. Examples of analgesics that can be used with the compositions, methods, and kits of the present invention to reduce discomfort due to inflammation include, but are not limited to, lidocaine, mepivacaine, bupivacaine, procaine, chloroprocaine, etidocaine, prilocaine dyclonine, hexylcaine, procaine, cocaine, ketamine, morphine, pramoxine, propophol, phenol, naloxone, meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin(diacetylmorphine), hydrocodone(dihydrocodeinone), hydromorphone(dihydromorphinone), levorphanol, meptazinol, methadone, metopon(methyldihydromorphinone), nalbuphine, oxycodone(dihydrohydroxycodeinone), oxymorphone(dihydrohydroxymorphinone), phenadoxone, phenazocine, remifentanil, tramadol, tetracaine, and mixtures thereof, as well as pharmaceutically acceptable salts and esters thereof. In preferred embodiments, a composition includes an analgesic selected from the group consisting of lidocaine, hydromorphone, oxycodone, morphine and pharmaceutically-acceptable salts thereof.

Antibiotics. Antibiotics may be used with the compositions, methods, and kits of the present invention, such as including, but not limited to Acrofloxacin, Amoxicillin plus clavulonic acid (i.e., Augmentin), Amikacin, Amplicillin, Apalcillin, Apramycin, Astromicin, Arbekacin, Aspoxicillin, Azidozillin, Azithromycin, Azlocillin, Bacitracin, Benzathine penicillin, Benzylpenicillin, Carbencillin, Cefaclor, Cefadroxil, Cefalexin, Cefamandole, Cefaparin, Cefatrizine, Cefazolin, Cefbuperazone, Cefcapene, Cefdinir, Cefditoren, Cefepime, Cefetamet, Cefixime, Cefmetazole, Cefminox, Cefoperazone, Ceforanide, Cefotaxime, Cefotetan, Cefotiam, Cefoxitin, Cefpimizole, Cefpiramide, Cefpodoxime, Cefprozil, Cefradine, Cefroxadine, Cefsulodin, Ceftazidime, Ceftriaxone, Cefuroxime, Chlorampenicol, Chlortetracycline, Ciclacillin, Cinoxacin, Ciprofloxacin, Clarithromycin, Clemizole penicillin, Clindamycin, Cloxacillin, Daptomycin, Demeclocycline, Desquinolone, Dibekacin, Dicloxacillin, Dirithromycin, Doxycycline, Enoxacin, Epicillin, Erthromycin, Ethambutol, Fleroxacin, Flomoxef, Flucloxacillin, Flumequine, Flurithromycin, Fosfomycin, Fosmidomycin, Fusidic acid, Gatifloxacin, Gemifloxaxin, Gentamicin, Imipenem, Imipenem plus Cilistatin combination, Isepamicin, Isoniazid, Josamycin, Kanamycin, Kasugamycin, Kitasamycin, Latamoxef, Levofloxacin, Lincomycin, Linezolid, Lomefloxacin, Loracarbaf, Lymecycline, Mecillinam, Meropenem, Methacycline, Methicillin, Metronidazole, Mezlocillin, Midecamycin, Minocycline, Miokamycin, Moxifloxacin, Nafcillin, Nafcillin, Nalidixic acid, Neomycin, Netilmicin, Norfloxacin, Novobiocin, Oflaxacin, Oleandomycin, Oxacillin, Oxolinic acid, Oxytetracycline, Paromycin, Pazufloxacin, Pefloxacin, Penicillin G, Penicillin V, Phenethicillin, Phenoxymethyl penicillin, Pipemidic acid, Piperacillin, Piperacillin and Tazobactam combination, Piromidic acid, Procaine penicillin, Propicillin, Pyrimethamine, Rifabutin, Rifamide, Rifampicin, Rifamycin SV, Rifapentene, Rokitamycin, Rolitetracycline, Roxithromycin, Rufloxacin, Sitafloxacin, Sparfloxacin, Spectinomycin, Spiramycin, Sulfadiazine, Sulfadoxine, Sulfamethoxazole, Sisomicin, Streptomycin, Sulfamethoxazole, Sulfisoxazole, Synercid (Quinupristan-Dalfopristan combination), Teicoplanin, Telithromycin, Temocillin, Tetracycline, Tetroxoprim, Thiamphenicol, Ticarcillin, Tigecycline, Tobramycin, Tosufloxacin, Trimethoprim, Trimetrexate, Trovafloxacin, Vancomycin, and Verdamicin or other known antibiotics.

f. Pigments. Pigments may be used either to add pigmentation, such as for cosmetic purposes. Pigments may be added in order to cover up other pigments, such as tattoo hiding. The present compositions may be sufficiently dense and non transparent so that skin-tone matching materials may be used to effectively cover up colored tattoos. Other purposes for pigmentation, such as treatment of vitilago or other skin coloration issues may be treated with the present compositions and methods containing suitable pigmentation. Pigments may be added further cosmetic purposes or for restorative tissue augmentation.

g. Enzyme Inhibitors or Enzymes. Enzyme inhibitors which would tend to prevent degradation of relevant constituents may be included, e.g., for example, protease inhibitors capable of inhibiting collagenase activity, and hyaluonidase inhibitors capable of inhibiting hyaluronidase activity. Alternatively, if controlled biodegradability is desired, enzyme inhibitors for controlled degradation may be included in such a way to have a particular sustained release profile (e.g., encapsulation within a sustained release vehicle, as part of the tissue augmentation composition). In a further alternative method, selective degradation of a tissue augmentation can be initiated by the use of a biologically compatible degradation agent, such as an enzyme which would hasten biodegradation.

h. Combination of Materials. Additionally, a patient may use a combination of materials for tissue augmentation, including silicone or other pre-solidified polymers, along with the present compositions and methods. A number of such materials can be found for example in Baumann, Cosmetic Dermatology, Principles & Practice (2002, McGraw-Hill, New York, ISBN 0-07-136281-9) at Chapter 19, pp. 155-172.

4. Polymerization, Cross-Linking and Initiation Agents

While one may desire a moldable consistency upon initial injection using a mold to achieve a predetermined outcome, one will not typically want an augmented region to continue to be moldable after the clinician has completed the procedure. In situ cross linking (and/or polymerization) can be used to increase rigidity. Therefore, for facial or body sculpting of an individual, the crosslinking and/or polymerization is able to take place in physiologic conditions.

Suitable cross linking conditions will be chosen based on the chemical structure of the monomers to be polymerized, the desired mechanical and persistence properties of the hydrogel after polymerization, and other considerations as described below and further known in the art. In some embodiments, cross-linking occurs by irradiation with a light at a wavelength of between about 100-1500 nm, and if in the long wavelength ultraviolet range or visible range, 320 nm or higher, and may be at about 514 or 365 nm. In some embodiments, cross-linking occurs at temperature in the physiologic range (e.g., about 37° C.) and in some embodiments at temperatures warmer or cooler, such as temperature on the surface or just below the surface of the skin, or at a predetermined temperature depending on the initiator used and the desired outcome. In some embodiments, cross-linking is chemically activated by a chemical activator (rather than a photoactivator) to trigger the polymerization of monofunctional, heterobifunctional, and homo-bifunctional cross-linkers. For example, heterobifunctional crosslinking moieties may be selected from among cross-linkers having at one reactive end an NHS ester or other active ester functionality, and a sulfhydrylreactive group on the other end. The sulfhydryl-reactive groups may be selected from, for example, maleimides, pyridyl disulfides and α-haloacetyls. Numerous other sulfhydryl reactive moieties are well known in the art; any suitable sulflhydryl reactive moiety can be used. Further, other orthogonally reactive species are known in the art and can be chosen for use in heterobifunctional crosslinking agents of the invention. In some embodiments, polymerization occurs at conditions such as at body temperatures, suitable chemical moiety interaction conditions, and suitable light conditions.

In general, hydrogels involve a hydrophilic backbone functionalized for cross-linking to form an interpenetrating network. Regardless of the monomer, upon polymerization, the material will have a more solid consistency in situ due to the presence of cross-linkages. Cross-linking results in increased solidifying (or gelation), and the more cross-linkages among molecules comprising the hydrogel, the more “solid” the hydrogel will become.

One may selectively solidify only partially either before or after administration into or upon the tissue to be augmented. It may be advantageous to have a moldable, or viscous fluidic material for administration, so that the material is pliable but not totally amorphous, under a mold, as contemplated herein.

The degree and type of cross-linkages may be manipulated to adjust the physical characteristics of the polymerized material. Also, some of the monomers that can be used (e.g., PEG) can be of various lengths/masses, and these too can be varied to alter the physical characteristics of the material after polymerization.

Cross-linking is the process of chemically joining two or more molecules by a covalent bond. As is well known in the art, cross-linking reagents contain reactive ends to specific functional groups (primary amines, sulfhydryls, etc.) on proteins or other molecules. Cross-linkers can be homobifunctional or heterobifunctional. Homobifunctional cross-linkers have two identical reactive groups. Heterobifunctional cross-linkers possess two different reactive groups that allow for sequential (two-stage) conjugations, helping to minimize undesirable polymerization or self-conjugation. Often different spacer arm lengths are required because steric effects dictate the distance between potential reaction sites for cross-linking.

One may select the type of cross linking reagent desired. For example, one may select a heterobifunctional crosslinking moiety for use with a temperature sensitive reactive group and a photosensitive reactive group.

a. Chemical Crosslinking. Chemical cross-linking can be accomplished by a number of means including, but not limited to, chain reaction (addition) polymerization, step reaction (condensation) polymerization and other methods of increasing the molecular weight of polymers/oligomers to very high molecular weights. Chain reaction polymerization includes, but is not limited to, free radical polymerization (thermal, photo, redox, atom transfer polymerization, etc.), cationic polymerization (including onium), anionic polymerization (including group transfer polymerization), certain types of coordination polymerization, certain types of ring opening and metathesis polymerizations, etc.

Step reaction polymerizations include all polymerizations which follow step growth kinetics including but not limited to reactions of nucleophiles with electrophiles, certain types of coordination polymerization, certain types of ring opening and metathesis polymerizations, etc. Other methods of increasing molecular weight of polymers/oligomers include but are not limited to polyelectrolyte formation, grafting, ionic cross-linking, etc.

Within the hydrogel, various cross-linkable groups are known to those skilled in the art and can be used, according to what type of cross-linking is desired. For example, hydrogels can be formed by the ionic interaction of divalent cationic metal ions (such as Ca²⁺ and Mg⁺²) with ionic polysaccharides such as alginates, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, and amylopectin. Multifunctional cationic polymers, such as poly(1-lysine), poly(allylamine), poly(ethyleneimine), poly(guanidine), poly(vinyl amine), which contain a plurality of amine functionalities along the backbone, may be used to further induce ionic cross-links.

Hydrophobic interactions are often able to induce physical entanglement, especially in polymers, that induces increases in viscosity, precipitation, or gelation of polymeric solutions. Block and graft copolymers of water soluble and insoluble polymers exhibit such effects, for example, poly(oxyethylene)-poly(oxypropylene) block copolymers, copolymers of poly(oxyethylene) with poly(styrene), poly(caprolactone), poly(butadiene), etc.

Solutions of other synthetic polymers such as poly(N-alkylacrylamides) also form hydrogels that exhibit thermo-reversible behavior and exhibit weak physical cross-links on warming. A two component aqueous solution system may be selected so that the first component (among other components) consists of poly(acrylic acid) or poly(methacrylic acid) at an elevated pH of around 8-9 and the other component consists of (among other components) a solution of poly(ethylene glycol) at an acidic pH, such that the two solutions on being combined in situ result in an immediate increase in viscosity due to physical cross-linking.

Other means for polymerization of the monomers also may be advantageously used with monomers that contain groups that demonstrate activity towards functional groups such as amines, imines, thiols, carboxyls, isocyanates, urethanes, amides, thiocyanates, hydroxyls, etc., which may be naturally present in, on, or around tissue.

Another strategy is to cross link by removal of protective groups which prevent cross linking. Thus, reactive groups may be present, but effectively chemically inhibited by means known in the art. Removal of these inhibiting groups would result in exposure of the reactive groups available for crosslinking. This removal may be done in situ in a human, such as by exposure to biocompatible reagents or conditions.

Alternatively, such functional groups optionally may be already provided in some of the monomers of the composition, so derivatizing to create reactive groups is not needed. In this case, no external initiators of polymerization are needed and polymerization proceeds spontaneously when two complementary reactive functional groups containing moieties interact at the application site.

Desirable cross-linkable groups include (meth)acrylamide, (meth)acrylate, styryl, vinyl ester, vinyl ketone, vinyl ethers, etc. In some embodiments, ethylenically unsaturated functional groups may be used. Other kinds of cross-linking with or without chemical bonding can be initiated by chemical mechanisms or by physical mechanisms. Cross-linking, in situ or otherwise, can be accomplished mechanically, for example, by interconnecting mechanically.

Cross-linkages may be formed via the innate chemical compositions of the fluidic tissue augmentation material. whereupon exposure to low-intensity 365 nm UV light and in the absence of photoinitiators or catalysts, gelatin having p-nitrocinnamate pendant groups (Gel-NC) may cross-link within minutes into a gelatin-based hydrogel as monitored by UV-vis spectroscopy.

b. Photoinitiation. The tissue augmentation composition may contain functionalized moieties allowing for light activated cross-linking, herein also referred to as “photoinitiated” or “photopolymerization”. To initiate the cross-linking reaction, a single electron chemical species known as a ‘radical’ must be created, either using a photo-initiator (which forms radicals after illumination with light of the proper wavelength). The radical then can transfer its unstable single electron species to one of the chemically reactive groups, causing that reactive group to become reactive. A radicalized group can then become more energetically stable by reacting with a non-radicalized group, thus forming a covalent bond. A chain reaction can then proceed in which the radical is transferred from bond to bond, causing a rapid formation of a polymer network out of the monomers in solution.

Photoinitiator moieties may be selected from long-wave ultra violet (LWUV) light-activatable molecules as are well known in the art, e.g. for example 4-benzoylbenzoic acid, [(9-oxo-2-thioxanthanyl)-oxy]acetic acid, 2-hydroxy thioxanthone, and vinyloxymethylbenzoin methyl ether; visible light activatable molecules such as acridine orange, ethyl eosin, eosin Y, Eosin B, erythrosine, fluorescein, methylene green, methylene blue, phloxime, riboflavin, rose bengal, thionine, and xanthine dyes, and thermally activatable molecules such as 4,4′ azobis(4-cyanopentanoic) acid and 2,2-azobis[2-(2-imidazolin-2-yl)propane]dihydrochloride.

For human skin, light in the 400-550 nm visible spectrum penetrates more effectively than light in the ultraviolet portion of the EM spectrum. Therefore, for transdermal photoinitiation of cross-linking, particularly hydrogels, for tissue augmentation, one may select between about 400 to about 550 nm as a desired initiation wavelength, with the term “about” indicating the range of light that can penetrate the subject human skin sufficiently to reach the hydrogel (or other fluidic tissue augmentation material) for cross-linking. One may also try to use wavelengths with inferior skin penetration properties (e.g., UV light), but it is anticipated that despite the higher photonic energies of these wavelengths, extended illumination may be required do to the fact that human skin absorbs the majority of UV photons.

For example, polyethylene oxide dimethacrylate hydrogels for photoinitiated cross-linking can be prepared by mixing poly(ethylene glycol)diacrylate (e.g., PEGDA; Nektar (previously Shearwater) Corporation, Huntsville, Ala., USA) in sterile phosphate buffered saline. A photoinitiator, e.g., Igracure 2959(Ciba Specialty Chemicals Corporation, Tarrytown, N.Y., USA), may be added for photoinitiation at long wave 365 nm UV light. Optionally, the tissue surface may be prefunctionalized so that the hydrogel polymerization reaction results the formation of covalent bonds between a surface and the hydrogel network. Preferably, for use in humans and animals, the photoinitiator is not toxic.

One may seek to deliver the light to the depth at which the fluidic tissue augmentation is located via, for example, fiber optic means, such as arthroscopically. One may insert fiber optic or other light source simultaneously or sequentially with fluidic tissue augmentation material administration by injection, for example of the fluidic tissue augmentation material.

B. Mold for Three-Dimensional Shape

The present invention also provides means for use of a mold to predetermine the shape of the present tissue augmentation materials. (The term “mold” is used herein to denote a structural guide, which may be made of any suitable material, for example, plastic, that is applied to the outside of the skin such that a fluid injected into the skin/dermis would expand the skin into the cavity created by the mold, thereby guiding the shape of the fluid in situ prior to photopolymerization). Although, as noted above, implants have been molded ex vivo to achieve a desired shape, the final shape of the augmented tissue is not predetermined. Because facial geometry is particularly complex, use of a mold to ensure natural-looking, aesthetically pleasing facial geometry is particularly needed in the tissue augmentation field. As indicated above, because the present tissue augmentation materials may be solidified (or selectively solidified to a predetermined solid or gelled state) in situ, placing a mold over the injection area allows a provider to predetermine the shape of the augmented tissue using injection molding techniques.

1. Transparency for Transdermal Photoinitiated Polymerization

If the injected dermal filler/tissue augmentation material is to be cross-linked using light, then the mold should be transparent to the wavelength of light to be used to activate polymerization. For transdermal polymerization, the light will travel through the skin to polymerize the injected filler. For example, a visible wavelength of about 400 nm to about 550 nm effectively penetrates human skin (the wavelength will depend on the quality of subject human skin), and therefore consideration must be given in selection of photoactivatable functionalities to ensure transmission of these wavelengths through a mold, skin, and/or underlying tissue. While UV light that has a wavelength that does not appreciably penetrate human skin, UV photons are excellent for initiating photochemistry. Therefore, there may be circumstances (e.g., areas in which the skin does not meaningfully attenuate UV light penetration) in which UV will be used, and therefore, circumstances in which the mold will need to be transparent to UV photons. One may test empirically any particular material for light wavelength transmission. Further, given that human skin is generally from about 1 mm to about 4 mm thick, one may further empirically test the transmission though to the subcutaneous tissue where the injected material would be polymerized.

If the light used to initiate cross-linking is delivered via a photo-catheter, the mold used need not be transparent to permit transdermal photoinitiated cross-linking.

One may pre-prepare a standardized mold against which the injectable tissue augmentation filler will be sculpted via in situ cross-linking of the soluble monomers into an insoluble polymer matrix. For example, for chin augmentation, a small double-hump mold (i.e., a plastic mold in the shape of a human chin including the cleft between the two symmetrical fat pad prominences) may be needed, and a physician may have on hand a library of standardized shapes, selecting the one that most closely matches the desired shape to be engineered on the patient's face.

For more customized applications, however, a custom-designed mold may be pre-prepared based on the patient.

One may use standard “mold making” techniques (using alginate, for example) to make a “negative” mask of a body surface to be augmented, then make a “positive” from the mask (which looks like the patient's face prior to the aesthetic correction) using clay or other sculpting material, for example, and alter that “positive” to make new ‘edited’ surface that contains the new shape to be placed on the patient's body surface (e.g., their face) during the aesthetic correction. A final “negative” mold is then manufactured using the altered “positive” as a guide. This final mold can be used to control the shape of an aesthetic correction. If transdermal photoinitiated cross-linking is used, the final mask should be transparent to the photons used for photoinitiation.

2. Computer Imaging Programs

Computer imaging programs can digitize the three-dimensional coordinates of surfaces, such as a body surface that is to receive a tissue augmentation/dermal filling procedure, and, a computer based program can be used to alter the digital information to conform to the desired outcome for the three-dimensional surface facial tissue after performance of an aesthetic correction, for example. Computer aided design (“CAD”) is used to construct a digital model of the treated body surface. These data can be used for rapid prototyping: using the three-dimensional data to create a plastic mold in the shape of the “negative”, or concavity, of the patient's desired features.

Three-dimensional coordinates may be obtained via scanning the surface. The scanner may be optical, such as a laser, or other type, such as tactile or acoustic. Ideally, scan resolution is in the 10 micron to 100 micron range to ensure authenticity in the detailed mold, and therefore predictable result of treatment. Regardless of the type of scanner, the data are in computer storable form, e.g., digitized, as 3 dimensional coordinates (“3D coordinates”). Typically, a computer program will obtain the information from the scanning source, and digitize such information into three coordinates, the x, y, and z axis. These fields reflect the location of that point in space, e.g., height, length, and depth relative to other points on the scanned surface.

Computer programs exist for photogrammetry, e.g., digitizing the photographic coordinates of a three-dimensional surface. For the three-dimensional coordinates of the surface of a person (or other irregularly shaped object), these programs are available in the area of biometrics in the security and law enforcement area. In forensic sciences, for example, “morphological fingerprints” record the three-dimensional of a surface, such as a crime victim with wounds. CAD, computer aided design, can match up the three-dimensional aspect of a wound with the suspected weapon, to determine if the wound could have been caused by the weapon.

Additional data may be recorded and merged with the three-dimensional coordinates of the desired surface shape. For example, a provider may wish to have a gradient of different densities of polymeric material layered upon the bone, with the most inflexible polymeric material closest to the bone, and the more plastic/skin like material closer to the surface of the skin. Internal information, obtained non-invasively and optionally in computer readable form, may allow for algorithms which set forth formulas for cross-linking polymers at different densities. Internal information may permit zonal fluidic tissue augmentation in that zones deepest within the body may permit different compositions, such as those with particulate matter, which would be unsuited for areas closer to the surface of the skin.

This internal and external information may be used to estimate the volume of injected liquid needed to mediate the aesthetic correction. Or such information may be used to determine the depth to which the liquid filler will be injected, and adjust the cross-linking-initiation wavelength. For example, radiological data volume scan (e.g., CT, MRI), may be used to determine bone depth or internal wound shape if the tissue augmentation is for tissue reconstruction after wounding.

Commercial suppliers of biometric computer programs include A4 Vision Inc., 840 West California Ave. Suite 200 Sunnyvale, Calif. 94086. Other commercial suppliers of computer programs that allow for digitizing the 3-D morphological information of a person are in the animation area. In this area, a three-dimensional object is “rendered” into digital information, and a computer program essentially “fills in” information based on algorithms pertaining to lighting, shading, motion, and any other parameters in the user interface. For example, Pixar Animation Studios, Emeryville Calif., offers a photorealistic rendering program for use by animators and others.

3. Tangible Mold Manufacturing

Rapid prototyping technology may be used to prepare a tangible mold. This is generally performed by a computer based method for using the three-dimensional coordinates for controlling a device which will prepare a mold in accordance with the three-dimensional coordinates. This is done generally by ink-jet or other deposition technology. Preferably, for transdermal photoinitiated cross-linking of tissue augmentation compositions, the mold will allow passage of for the light used for initiation of cross-linking, and therefore be transparent to appropriate wavelengths. To allow for the injection of fluidic tissue augmentation materials, the mold will have small apertures or be sufficiently soft to allow a needle injector (or other device) to penetrate through the mold for application/injection of the fluidic tissue augmentation composition. The concavity of the mold will be in the shape of the desired outcome for the tissue augmentation.

4. Virtual Mold

Non-tangible information may be used to guide tissue augmentation in situ. Computer readable digital information may be visualized in any number of ways. The provider may use electronic guides, such as use of electronic indicators during tissue augmentation procedure, such as laser or other light indicators.

III. Methods of Preparing and Using Tissue Augmentation Compositions

The material contemplated for the present injectable tissue augmentation compositions and methods is to be sufficiently amorphous to be placed within a body without the need for invasive surgical techniques, such as those required by a solid implant. Fluidic or liquid material may be somewhat gelled or pasty, and capable of taking a shape when molded. Preferably, the composition is injectable using conventional syringe apparatus or other syringe-type apparatus involving a medically acceptable needle for subcutaneous injection. In some instances, the present compositions preferably will maintain their overall integrity, e.g., pre-solidified polymeric structure, even after injection, and not be subject to losing integrity due to mechanical shear forces of going through a needle.

A. Solidifying Time

In order for the tissue augmentation material to maintain the desired final shape, the present invention provides for selective solidifying in situ under physiologic conditions. Ideally, only one component will solidify under a prescribed set of conditions (e.g., exposure to light) so that undesired solidifying, i.e., cross linking to a second component of the composition, will be minimized. Solidification of the present compositions does not require total solidification. Where solidification results in a gel, it may be elastic or brittle.

In general, for human facial sculpting, one will select a composition/solidifying system which allows for increased solidity in a matter of minutes or less. In the methods of the invention, the time to achieve increased solidification is less than 10, 9, 8, 7, 6, 5, 4, 3, 2 minutes and may be less than one minute.

The time of solidification will generally be affected by, or can be modified by, changing at least the following variables: the polymerization initiator system, cross-link density, chemical reactivity of the reactive cross-linkable groups on the monomer, the monomer molecular weight, and monomer concentration in solution. A higher cross-link density will generally accelerate the process of solidifying, thereby reducing time; a lower molecular weight will provide a slower time. A higher monomer concentration will accelerate the process.

B. Varying Mechanical and Persistence Properties of the Tissue Augmentation Compositions

The persistence and mechanical properties may be varied depending on the particular application of the present tissue augmentation compositions.

C. Varying the Persistence Properties

One may select the persistence properties of the tissue augmentation composition by controlling its rates of (a) mechanical dispersion and (b) chemical degradation. The monomers or polymeric subunits that form the subject hydrogel may be constructed so that the overall tissue augmentation material is degradable. Ideally, for human or animal use, upon degradation in situ, the degradation products will not cause adverse effects.

Controllable degradation or erosion is also important because the surrounding tissues change over time. Thus, while tissue augmentation at one age may appear natural, the surrounding tissue may change in appearance, thus altering the appearance of the tissue so augmented. Bone may undergo degradation. Surrounding muscle may become stretched or depleted, and one may choose to administer compositions which prevent muscle degradation or promote muscle growth. Such compositions may be co administered, or administered in seriatim over a period of time, so that the tissue augmentation material continues to appear natural because the surrounding tissue has not changed substantially.

The controllable erosion profile may be used for drug delivery, for example. One may wish to administer compositions that prevent or reverse bone degradation, such as osteoclast blocking agents or osteoblast promoting agents.

The chemical structure of the hydrogel may be designed to possess specific degradative properties, both in terms of extent of degradation (i.e., complete or partial) and in terms of time to complete or partial degradation.

Biodegradable hydrogels can be composed of polymers or monomers covalently connected by linkages susceptible to biodegradation, such as ester, acetal, carbonate, peptide, anhydride, orthoester, phosphazine, and phosphoester bonds.

For purposes of controllable erosion, one may prepare regions within the hydrophilic backbone which will dissociate or de-crosslink upon certain conditions, such as enzyme treatment.

One may include peptide or protein moieties for enzymatic degradation or other means for controlled durability. Enzymatically degradable linkages include poly(amino acids), gelatin, chitosan, and carbohydrates.

For example, the degradable region may be polymers and oligomers of glycolide, lactide, epsilon caprolactone, other hydroxy acids, and other biologically degradable polymers that yield materials that are non-toxic or present as normal metabolites in the body. Poly(alpha-hydroxy acids) are poly(glycolic acid), poly(DL-lactic acid) and poly(L-lactic acid).

Other useful materials include poly(amino acids), poly(anhydrides), poly(orthoesters), poly(phosphazines), and poly(phosphoesters). Polylactones such as poly(epsilon.-caprolactone), poly(epsilon-caprolactone), poly(delta-valerolactone) and poly(gamma-butyrolactone), for example, are also useful.

D. Thermoresponsiveness

The present hydrogels may be made to be thermoresponsive for example to degrade upon reaching a certain temperature. This may be useful for reversible persistence qualities, e.g., use of heat to “melt” a tissue augmentation composition of the present invention in situ so that one may “re-do” the injectable implant aspect as the face ages.

E. Varying the Mechanical Properties

The mechanical properties of the tissue augmentation material within a patient are a significant consideration. With a more flexible structure allowing for greater mechanical movement, the material may appear more natural than a less flexible material, in certain areas. A more flexible material, may, however, be less persistent and not have the desired duration within the body. One may select the desired combination of persistence and mechanical properties by selecting the composition and form of the materials.

One may seek to modulate the mechanical properties, such as tensile or shear strength by varying the structure of the monomers that polymerize to form the hydrogel, or the nature of the bonds used for cross-linkages between the monomers. One may choose to vary the composition to vary the mechanical properties. The firmness of the formed hydrogel will be determined in part by the hydrophilic/hydrophobic balance, where a higher hydrophobic percent provides a firmer hydrogel. The firmness will also be determined by the cross-link density (higher cross-link density produces a firmer hydrogel), the monomer molecular weight (lower MW provides a firmer hydrogel), and the length of the cross-link (a shorter cross-link produces a firmer hydrogel, using a crosslinking reagent linking arm may produce less rigidity). The swelling of the hydrogel is inversely proportional to the cross-link density. Generally, no or minimal swelling is desired, desirably less than about 10 percent. Elasticity of the formed hydrogel can be increased by increasing the size of the distance between cross-links and decreasing the cross-link density (e.g., by linker arm). Incomplete cross-linking will also provide a more elastic hydrogel. Preferably the elasticity of the hydrogel substantially matches the elasticity of the tissue into which the composition is inserted.

Varying the nature of the chemical bonds comprising the cross-links may confer different mechanical properties upon the hydrogel. For example, in some hydrogels, increasing the density of covalent cross-linkages may increase elasticity but produce a more brittle gel. Simultaneously increasing the density of ionic cross-linkages and the distance between cross-links may increase both elasticity and toughness. Ionic cross-links and their length may be important in dissipating the energy of deformation due to a partial and stepwise de-cross-linking. Covalently cross-linked gels may undergo energy accumulation and may therefore not be as elastic.

Within tissue, obstruction will limit movement. If tissue augmentation is in a relatively immobile location, e.g., the chin, then one mechanical property may be desired. If, however, the tissue augmentation is in an area subject to more frequent mechanical stress, such as the cheeks, the lip and mouth area or the eye area, then the final mechanical properties should be more elastic.

Thus, the present tissue augmentation compositions in areas not subject to relatively constant movement, such as over a cheek or chin bone, or on the bridge of the nose, may persist longer than those in locations which have no solid object preventing movement—such as in the lips or mouth area, or parts of the eye area.

F. Persistence Characteristics

The persistence characteristics may be related to the mechanical characteristics. For example, with time, the cross-linked hydrogel/dermal filler composition may become unstable, not resulting in a loss of volume, but rather in a change of shape driven by the application of continuous mechanical forces. This is termed “creep”: Creep is defined as the time-dependent strain g (t) developed by a sample when a stress s is applied.

The amount of creep depends on the compliance of the sample, J(t), which relates the stress to the strain as:
g(t)=sJ(t)

(strain over time)=(stress on compliance over time)

For a perfectly elastic material, the compliance is the inverse of the modulus (i.e., a less stiff material is more compliant). For most samples, however, the different time dependencies of these functions results in more complex relationships.

Creep results in no volume change and is merely a rearrangement of the material. The very nature of polymeric components enables inter-chain motion, and subsequently some flow, when enough energy is introduced into the polymeric system. Thus, creep cannot be totally eliminated. Methods exist for minimizing creep, however.

Alternatively, cross-linking will minimize bulk chain motion that can lead to creep, effectively reducing the compliance (elasticity) over time. A cross-linked system will creep initially as the polymer molecules attempt to flow under the influence of an applied load, effectively rearranging the entangled nest of molecules. Once they are stretched taut against the cross-links, however, no further flow is possible, and creep stops.

Some investigators report routine observance in cross-linked polymeric implants, where initial creep is observed in the first year of in vivo service, often termed a “bedding-in” period, but no further creep is observed after this point. Wear, however, may continue, and often manifests itself as creep. Wear does result in volume loss, however.

The plasticity (solidity) of an injectable solution can be predetermined using a rheometer, which measures “hardness”. Different parts of the body will require more or less plasticity/solidity. As such, compositions with greater amounts of crosslinking will be designed for regions that require greater solidity (e.g., noses) versus regions that require softer tissue (e.g., breasts or cheeks).

IV. Methods of Cosmetic and Therapeutic Use

The present compositions and methods may be used in general to reshape tissue for cosmetic purposes. Tissue augmentation includes, but is not limited to, the following: dermal tissue augmentation, filling of lines, folds, wrinkles, minor facial depressions, cleft lips and the like, especially in the face and neck; correction of minor deformities due to aging or disease, including in the hands and feet, fingers and toes; augmentation of the vocal cords or glottis to rehabilitate speech; dermal filling of sleep lines and expression lines; replacement of dermal and subcutaneous tissue lost due to aging; lip augmentation; filling of crow's feet and the orbital groove around the eye; breast or penile augmentation; chin augmentation; augmentation of the cheek and/or nose; filling of indentations in the soft tissue, dermal or subcutaneous, due to, e.g., overzealous liposuction or other trauma; filling of acne or traumatic scars and rhytids; filling of breasts and/or buttocks; filling of nasolabial lines, nasoglabellar lines and infraoral lines. Also included is augmenting bone, such as facial bones either for cosmetic or reparative purposes, and cartilage, such as augmenting nasal cartilage for cosmetic or reparative purposes.

For example, one may desire tissue augmentation where hard or gel silicone implants would otherwise be used for aesthetic purposes such as the chin, cheek, nose, jaw, breast, pectoral areas, and legs/calves. Augmentation to portions of the lip where a more solidified tissue augmentation material may be desired is also contemplated.

One may desire tissue augmentation to counteract the signs of aging, such as facial wrinkles, loose skin, and bone and muscle mass loss.

Therapeutic purposes include lipoatrophy, a type of lipodystrophy involving fat loss rather than additional fat tissue, is a disorder caused by a thinning of fatty tissue and is often, but not solely, connected to Highly Active Antiretroviral Therapy (HAART) in HIV+ patients. The disorder is most visible in the facial areas (cheeks, eye sockets, temples), and often results in severe social stigmatization. The present invention is also useful in seroma prevention.

One may also use the present materials and methods for non-living subjects, e.g., in the aesthetic body reconstruction of a deceased body for interment or forensic purposes.

V. Kits

The present invention provides for kits for tissue augmentation. A first container comprises, consists essentially of, or consists of any of the compositions herein.

For example, a first container can comprise, consist essentially of, or consist of a tissue augmentation material capable of increasing solidity in situ under physiologic conditions, such as a hydrogel forming material containing moieties to allow transdermal photopolymerization.

This first container(s) may have sufficient volume to hold a size convenient for providers who augment tissue in the face, i.e. facial sculpting. For example, a first container may be adapted to hold a less than 500 mL, 100 mL solution, 20 mL solution 10 mL solution or 5 mL solution. For convenience of medical providers, this first container may be a syringe, referred to by manufacturers as a “prefilled syringe”, suitable for injecting the material into the tissue to be augmented.

The first container should preserve the integrity of the hydrogel forming composition, for example, by substantially preventing cross-linking. The first container may for example, be made of a light-impenetrable material so that a photoinitiated cross-linking reaction cannot be initiated.

The kit may contain a second container containing a dermal filler composition, such as those enumerated herein. For example, the second container may contain a hyaluronic acid composition, which is substantially incapable of cross-linking with the composition in the first container. The second container may be a prefilled syringe.

The kit may be used for injectable tissue augmentation either by premixing the composition in the first container with the composition in the second container, mixing in a third vessel which may be provided in the kit, or by injecting in seriatim into the same space within the tissue. The provider may “tune” or vary the mechanical or persistence properties by altering the ratios of the first composition to the second composition, either by premixing and then applying (e.g., injecting) or by applying in seriatim (e.g., two injections in the same location).

A kit according to the present invention may comprise a) a first prefilled syringe containing a photopolymerizing hydrogel moiety; and b) a second prefilled syringe containing a dermal filler, and optionally a transparent mold wherein the concavity in the mold is in the shape of a body part.

VI. Business Methods

Methods and the kits disclosed herein can be used to perform business services and/or sell business products.

In some embodiments, the present invention contemplates a business method that provides a kit and treatment services. For example, the business can make a formulation based on the compositions described herein. The business method herein can then manufacture a kit containing the formulations as disclosed herein. The business may further sell the kit for treatment. In some embodiments, the business method licenses a third party to manufacture the kit. In some embodiments, a business method of the present invention commercializes the kit disclosed herein. In any of the embodiments herein, the kit is optionally disposable.

The business method contemplates providing a treatment service in exchange for a service fee. The service can be provided directly to the patient by a health care provider.

The business method contemplates a computer-based method of providing a customized tissue augmentation kit for a provider of tissue augmentation services.

The present invention also includes a business method for providing a customized tissue augmentation kit for a particular patient including

transmitting to a receiving computer a computer file containing three dimensional coordinates of the desired shape of tissue-augmented area of a particular patient; and a computer file containing the desired mechanical and persistence properties of the tissue augmentation material;

wherein such information is used to prepare a customized tissue augmentation kit for use by a provider on the particular patient. The kit so provided contains a mold for the predetermined tissue augmented shape, an injectable tissue augmentation material capable of selectively solidifying in situ and having preselected persistence and mechanical properties in accordance with the computer file so transferred.

Other embodiments contemplate business methods for a financial rewards program based on usage of the compositions and methods herein. This is particularly useful in the area of cosmetic dermatology where patients pay for medicaments typically without any form of insurance or governmental reimbursement. Therefore, the pricing sensitivity for patients is important. For example, the business method may further include storing a computer file of the number of tissue augmentation purchases or services, and means for correlating this number with a financial discount program, and optionally further correlating this with a purchase price for the patient or provider.

### EXAMPLES

The following examples are provided to more precisely define and enable the compositions and methods of the present invention. It is understood that there are numerous other embodiments and methods of using the present invention that will be apparent embodiments to those of ordinary skill in the art after having read and understood this specification and examples. The following examples are meant to illustrate one or more embodiments of the invention and are not meant to limit the invention to that which is described below.

### Example 1

### Computer Aided Design of A Mold For Tissue Augmentation And Tissue Augmentation Of A Nose Via In Situ Polymerization According To The Shape Of The Mold

This prophetic example is to illustrate the preparation and use of a mold for tissue augmentation to obtain a predetermined result for tissue augmentation using an injectable dermal filler which can be cross-linked in situ. The use of a pre-formed mold can be performed in a step-wise fashion as described herein, where a patient desires tissue augmentation to his nose:

Step #1: Obtain the current 3D spatial coordinates of the tissue to be altered. The surface to be altered (e.g., a patient's nose) is scanned using an optical scanning device. The device records the three-dimensional coordinates of the nose (for example) in a data set communicated to a computer apparatus. As set forth above, other means of obtaining computer readable (e.g., digital) information regarding the contours of a tissue are available, such as acoustic or tactile, or photoprogrammic (using a photographic lens and light information to convert the entire three-dimensional structure at once, e.g., biometric computer applications).

Step #2: A computer readable data file is created. The data set of three-dimensional coordinates of the patient's nose is stored in terms of its spatial position (e.g., an “x” field for vertical height, a “y” field for horizontal length, and a “z” field for depth). The acquired image is stored as 3D coordinates in a data file.

Step #3: CAD is used to alter the stored 3D coordinates to reflect the desired shape of the tissue to be altered. With current computer aided design programs (“CAD”), the current 3D digitized coordinates can be visualized as the current shape of the tissue. The practitioner may, optionally in consultation with the patient, alter the 3D coordinates to reflect a desired shape of the tissue surface after tissue augmentation. For example, if a nose shape is flat and a patient desires a higher nasal bridge, the patient may select the ultimate shape of the nose including tissue augmentation materials.

Step #4: A mold (representing a ‘negative’ of the 3D shape of the aesthetic correction) of the desired outcome is fabricated in accordance with the data of the altered 3D coordinates. The computer based 3D coordinates are used to fabricate a mold using a rapid prototyping printer according to the contents of data file. The mold concavity will be the predetermined shape of the tissue after augmentation. If desired, the mold will be transparent in that it will allow transmission of photons of the appropriate wavelength, such as visible light wavelength of between about 400 and about 550 nm, for photoinitiated cross-linking of the injected tissue augmentation material. If photoinitiation is via fiber optic subdermal delivery of light, the mold need not be transparent. Small holes allows for the practitioner to put a needle through the mold to perform the injection of the tissue augmentation material into an appropriate area of the tissue. In the present prophetic example of augmenting bridge of the nose, the holes may be in a location on the bridge of the nose.

Step #5: The mold is used for tissue augmentation. The mold is held against the face with optionally suitable mechanical clamps or adhesive material. The mold should adhere to the surface tightly enough to form a concavity that will hold the to-be-injected material in place during the injection and subsequent cross-linking.

Step #6: Inject the fluidic tissue augmentation material through the delivery holes into the tissue, so that the tissue augmentation shape fits the internal concavity. The material is injected until the material causes the skin to ‘bleb’ out and to thus come into direct contact with the walls of the transparent mold. For the nose, where there is typically insufficient tissue to “bleb” out of the holes for injection, the patient's nose with fluidic tissue augmentation material will fit precisely within the mold. For example, a fluidic hydrogel precursor, derivatized for ultraviolet activated cross-linking and thus will solidify upon exposure to suitable wavelengths of light, can be admixed with a biocompatible dermal filler material, such as a type of collagen, hyaluronic acid or a silicone-containing material. The biocompatible dermal filler, such as the silicone containing material, is fluidic, yet does not chemically react with components of the hydrogel upon photoinitiation.

Step #7: Once the fluidic tissue augmentation material is injected, gelation/solidifying is initiated via cross-linking in situ. If transdermal photoillumination is used to initiate polymerization, a suitable external light source is used to transdermally illuminate the injected material while the transparent mold is held in place. For example, if a hydrogel is derivatized for visible light photoinitiated cross-linking, a suitable visible light source is held against the mold, which is held against the face. The mold should be transparent to the light source used, in this case, such as a transparent mold capable of transmitting suitable wavelengths. Light may be delivered beneath the skin surface using known means, such as fiber optics or arthroscopically. In other circumstances, cross-linking may be initiated using other means, such as temperature, chemical initiators, or other means known in the art. Cross-linking may be by removing cross-linking inhibitors to selectively expose reactive groups present on the hydrogel forming material.

Step #8: Optionally, the injection and polymerization process can be repeated, or can be performed in stages to build up the underlying gelled/solidified material gradually. One may use, for example, material that is more solid closer to the bone. Closer to the skin surface, one may use compositions which are more elastic.

### Example 2

### Increasing the In Vivo Persistence of Restylane™, Hyaluronic Acid Dermal Filler in a Human

This working example is to illustrate the preparation of a tissue augmentation composition prepared by mixing Restylane™ (2% hyaluronic acid) with a solution of 20% polyethylene glycol diacrylate, followed by injection of the 1% PEG-DA and 2% hyaluronic acid mixture.

In injection of Restylane™ enables the aesthetic correction of the nasolabial folds that persists 4.5 months after injection. Preliminary data show that the persistence of hyaluronic acid dermal filler can be extended in rodents, when injection is followed by transdermal photoillumination of the tissue augmentation material.

Restylane™ (which has a toothpaste like consistency) is combined 20:1 (Restylane™ volume to PEG-DA volume) with a 20% polyethylene glycol diacrylate (“PEG diacrylate”) solution (which has a water-like consistency), where the PEG-diacrylate is substantially not cross-linked at the time of mixing and injection, but is capable of forming a chemically cross-linked interpenetrating covalent network in situ after photoillumination. The final mixture is 1% PEG-DA and 2% hyaluronic acid.

Various hydrogel forming materials may be used. The PEG-diacrylate moiety is used for illustration in this example. Presently, the acrylate-containing molecule is here referred to as “(x) acrylate” to indicate that it may be selected from among a variety of acrylate-containing molecules. The acrylate-containing molecule may be a methacrylate, a polymethacrylate, a dimethacrylate or any number of acrylate-containing molecules suitable for use in vivo in humans to form hydrogels.

The PEG-diacrylate composition is (as a consequence of containing two chemically reactive acrylate groups) capable of cross-linking upon photoinitiation, in the presence of UV light and an appropriate photoinitiator (e.g., Igracure) to generate the single electron radical required for initiating the polymerization reaction.

In the PEG-(x) acrylate molecule, the chain length of the polyethylene glycol moiety (the hydrophilic backbone present in each monomer) is of sufficient length to confer desired mechanical properties (e.g., stiffness) under physiological conditions. The acrylate groups are located on either end of the monomeric PEG-diacrylate molecule to enable covalent cross-linking.

The 1% PEG-DA, 2% hyaluronic acid is also mixed with a photoinitiator (e.g., Igracure). The material can be injected in vivo and polymerized by phototransillumination.

### Example 3

### Kit for Tissue Augmentation

A kit is provided containing a mold prepared from the 3D data file as described above, and a syringe of tissue augmentation material selectively formulated to have specific mechanical and persistence properties after polymerization of the monomers to form an interpenetrating covalent network.

The kit contains a prefilled syringe containing a substantially uncross-linked solution of 1% PEG-DA in which the acrylate groups on the PEG-DA molecules are capable of chemical cross-linking in situ in the presence of ultraviolet light and a photoinitiator. The kit includes a separate second container, containing an injectable dermal filler material comprising a hyaluronic acid, a chondroitin, or a collagen (or an analog, derivative, functional fragment or peptidomimetic thereof of any of the preceding), suitable for use in humans (e.g., Restylane™, Zyplast™). The hyaluronic acid or collagen-containing composition does not crosslink with the hydrogel composition upon initiation of chemical cross-linking.

While the preferred embodiments of the present invention have been shown and described herein, it will be obvious to those skilled in the art that such embodiments are provided by way of example only. Numerous variations, changes, and substitution will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A kit comprising
(a) a first prefilled syringe containing at least one photopolymerizing hydrogel forming moiety; and
(b) a second prefilled syringe containing at least one dermal filler, and optionally a transparent mold wherein the inner surface of the mold is in the shape of a body part.

2. The kit of claim 1 wherein the at least one photopolymerizing hydrogel forming moiety is selected from the group consisting poly(lactic acid) (PLA), poly(glycolic acid) (PGA), and their copolymers, poly(lactic-co-glycolic acid) (PLGA), Polyethylene glycol diacrylate (PEG-DA), poly(ethylene oxide) (PEO), Pluronic, (PEO-PPO-PEO), poly(ethylene oxide)diacrylate (PEODA), polyalkylene oxides, polyethylene glycols, polyethylene oxides, partially or fully hydrolyzed polyvinylalcohols, poly(vinylpyrrolidone), poly(t-ethyloxazoline), poly(ethylene oxide)-co-poly(propylene oxide) block copolymers (poloxamers and meroxapols), propylene glycol, trimethylene glycols, mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), polymaleic acid; polyacrylamide, poly(methacrylamide), poly(dimethylacrylamide), poly(N-isopropyl-acrylamide); poly(olefinic alcohol), poly(N-vinyl lactams), poly(N-vinyl caprolactam), polyethylene glycol/poly(N-isopropylacrylamide); polyoxazolines, poly(methyloxazoline), poly(ethyloxazoline), polyvinylamines, polyacrylamide (PAA), poloxamines, carboxymethyl cellulose, and hydroxyalkylated celluloses.

3. The kit of claim 2 wherein the at least one photopolymerizing hydrogel forming moiety comprises polyethylene glycol derivatized with diacrylate.

4. The kit of claim 1 wherein the at least one dermal filler is selected from the group consisting of collagens, hyaluronic acid, elastins, laminins, fibronectins, chondroitin sulfate, keratin sulfate, hyaluronic acid, heparan sulfate, chondroitin sulfate, amylose, maltodextrin, amylopectin, starch, dextran, heparin, dermatan sulfate, dextran sulfate, pentosan polysulfate, and chitosan.

5. The kit of claim 4 wherein the at least one dermal filler comprises hyaluronic acid.

6. The kit of claim 1 further comprising an initiator of crosslinking.

7. The kit of claim 6 wherein the initiator is eosin.

8. The kit of claim 1 wherein the at least one photopolymerizing hydrogel forming moiety is derivatized to selectively solidify in the presence of a light wavelength which substantially penetrates mammalian skin below the epidermis; and, the at least one dermal filler is substantially incapable of selective solidifying in the presence of a light wavelength which penetrates human skin.

9. The kit of claim 1 wherein the at least one photopolymerizing hydrogel forming moiety is selectively degradable in situ.

10. The kit of claim 1 wherein the mold is pre-prepared using a computer program capable of transmitting three dimensional coordinates of the predetermined shape to a device which prepares a tangible mold reflecting the three dimensional coordinates.

11. A method for augmenting tissue in a predetermined shape comprising
(a) injecting a moldable tissue augmentation composition into the tissue for which augmentation is desired;
(b) applying a mold externally to skin covering the tissue for which augmentation is desired, wherein an inner concave surface of the mold is in a predetermined shape wherein applying the tissue augmentation composition will bring the skin into contact with the inner concave surface of the mold; and,
(c) increasing the solidity of the tissue augmentation material whereby the tissue augmentation material holds the shape of the inner concave surface of the externally applied mold.

12. The method of claim 11 wherein the moldable tissue augmentation material comprises a hydrogel and a dermal filler.

13. The method of claim 11 wherein the dermal filler is chosen from the group consisting of collagens, hyaluronic acid, elastins, laminins, fibronectins, chondroitin sulfate, keratin sulfate, hyaluronic acid, heparan sulfate, chondroitin sulfate, amylose, maltodextrin, amylopectin, starch, dextran, heparin, dermatan sulfate, dextran sulfate, pentosan polysulfate, and chitosan.

14. The method of claim 13 wherein the dermal filler is hyaluronic acid.

15. The method of claim 11 wherein the wherein the hydrogel is selected from the group consisting of poly(lactic acid) (PLA), poly(glycolic acid) (PGA), and their copolymers, poly(lactic-co-glycolic acid) (PLGA), Polyethylene glycol diacrylate (PEG-DA), poly(ethylene oxide) (PEO), Pluronic, (PEO-PPO-PEO), poly(ethylene oxide)diacrylate (PEODA), polyalkylene oxides, polyethylene glycols, polyethylene oxides, partially or fully hydrolyzed polyvinylalcohols, poly(vinylpyrrolidone), poly(t-ethyloxazoline), poly(ethylene oxide)-co-poly(propylene oxide) block copolymers (poloxamers and meroxapols), propylene glycol, trimethylene glycols, mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose, polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), polymaleic acid, polyacrylamide, poly(methacrylamide), poly(dimethylacrylamide), poly(N-isopropyl-acrylamide), poly(olefinic alcohol), poly(N-vinyl lactams), poly(N-vinyl caprolactam), polyethylene glycol/poly(N-isopropylacrylamide); polyoxazolines, poly(methyloxazoline), poly(ethyloxazoline), polyvinylamines, polyacrylamide (PAA), poloxamines, carboxymethyl cellulose, and hydroxyalkylated celluloses.

16. The method of claim 15 wherein the hydrogel is polyethylene glycol diacrylate.

17. The method of claim 11 wherein the increasing the solidity of the tissue augmentation material is initiated by a light source

18. The method of claim 17 wherein the light source has a wavelength of 400-550 nm.

19. The method of claim 17 wherein the initiation by a light source is performed transdermally.

20. The method of claim 17 wherein the initiation by a light source is performed subdermally.

21. The method of claim 11 wherein the mold is pre-prepared using a computer program capable of transmitting three dimensional coordinates of the predetermined shape to a device which prepares a tangible mold reflecting the three dimensional coordinates.

22. The method of claim 11 wherein the applying of the mold to the skin is performed before applying the moldable tissue augmentation material.

23. The method of claim 11 wherein the applying of the mold to the skin is performed at the same time as applying the moldable tissue augmentation material.

24. A method for facial sculpting comprising
(a) predetermining the final shape of the sculpted face by using digital three dimensional information to prepare a mold having an inner surface comprising the precise dimensions of the final sculpted face;
(b) injecting tissue augmentation material capable of increasing in solidity in situ upon physiologically compatible initiation using the mold as a guide to form the shape of sculpted face; and
(c) applying the physiologically compatible initiation to increase the solidity of the tissue augmentation material to maintain the shape of the inner surface of the mold.

25. The method of claim 24 wherein the tissue augmentation material comprises a hydrogel and a dermal filler.

26. The method of claim 25 wherein the dermal filler is chosen from the group consisting of collagens, hyaluronic acid, elastins, laminins, fibronectins, chondroitin sulfate, keratin sulfate, hyaluronic acid, heparan sulfate, chondroitin sulfate, amylose, maltodextrin, amylopectin, starch, dextran, heparin, dermatan sulfate, dextran sulfate, pentosan polysulfate, and chitosan.

27. The method of claim 25 wherein the wherein the hydrogel is selected from the group consisting of poly(lactic acid) (PLA), poly(glycolic acid) (PGA), and their copolymers, poly(lactic-co-glycolic acid) (PLGA), Polyethylene glycol diacrylate (PEG-DA), poly(ethylene oxide) (PEO), Pluronic, (PEO-PPO-PEO), poly(ethylene oxide)diacrylate (PEODA), polyalkylene oxides, polyethylene glycols, polyethylene oxides, partially or fully hydrolyzed polyvinylalcohols, poly(vinylpyrrolidone), poly(t-ethyloxazoline), poly(ethylene oxide)-co-poly(propylene oxide) block copolymers (poloxamers and meroxapols), propylene glycol, trimethylene glycols, mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose, polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), polymaleic acid, polyacrylamide, poly(methacrylamide), poly(dimethylacrylamide), poly(N-isopropyl-acrylamide); poly(olefinic alcohol), poly(N-vinyl lactams), poly(N-vinyl caprolactam), polyethylene glycol/poly(N-isopropylacrylamide), polyoxazolines, poly(methyloxazoline), poly(ethyloxazoline), polyvinylamines, polyacrylamide (PAA), poloxamines, carboxymethyl cellulose, and hydroxyalkylated celluloses.

28. The method of claim 25 wherein the tissue augmentation material comprises polyethylene glycol diacrylate and hyaluronic acid.

29. The method of claim 24 wherein the physiologically compatible initiation is light.

30. The method of claim 29 wherein the light has a wavelength of 400-550 nm.

31. The method of claim 29 wherein the light is applied transdermally.

32. The method of claim 29 wherein the light is applied subdermally.

33. The method of claim 24 wherein the mold is pre-prepared using a computer program capable of transmitting three dimensional coordinates of the predetermined shape to a device which prepares a tangible mold reflecting the three dimensional coordinates.

34. The method of claim 24 wherein the applying of the mold to the skin is performed before applying the moldable tissue augmentation material.

35. The method of claim 24 wherein the applying of the mold to the skin is performed at the same time as applying the moldable tissue augmentation material.
